(19) United States
(12) Patent Application Publication
(10) Pub. No.: US 2010/0189597 A1
(43) Pub. Date: Jul. 29, 2010

(54) Methods and Systems for Preparing Blood Products

(75) Inventor: Dennis J. HLAVINKA, Arvada, CO (US)

Correspondence Address: CaridianBCT, Inc.;Mail Stop: 810 1F2, 10811 WEST COLLINS AVE, LAKEWOOD, CO, 80215, US

(73) Assignee: CaridianBCT Biotechnologies, LLC, Lakewood, CO (US)

(21) Appl. No.: 12/753,933

(22) Filed: Apr. 5, 2010

Related U.S. Application Data
(62) Division of application No. 11/688,694, filed on Mar. 20, 2007.

(51) Int. Cl. A61L 2/08

(52) U.S. Cl. 422/22

(57) ABSTRACT

The present invention is related to pre-connected disposable sets including a pre-connected bag containing a photosensitizer for use in a blood separation and pathogen inactivation procedure.

### PRIORITY

This application is a divisional of U.S. patent application Ser. No. 11/688,694 filed on Mar. 20, 2007.

### BACKGROUND

Whole blood collected from healthy donors is routinely separated into components: platelets, plasma and red blood cells. One or more of the separated components are used for later administration (transfusion) to a patient who may be in need of the particular component. For example, red blood cells may be administered to a patient to replace blood loss or to treat patients with chronic anemia. Plasma may be administered to treat clotting factor deficiencies. Platelets are commonly administered as a therapy to cancer patients whose ability to generate platelets has been compromised by chemotherapy.

There are traditionally two ways to obtain these blood components. One way is to collect whole blood from donors/patients and separate it into components manually at some time period after the whole blood collection.

Another way to separate whole blood into components is by using automated cell-separation devices. Apheresis machines are devices which automatically separates whole blood into components, and returns any uncollected blood components back to the donor during the collection procedure.

An alternative to manual processing of whole blood as described above is the automatic processing of whole blood using an automated device.

Whether whole blood is separated manually, by apheresis, or by automatic processing, before transfusion to a patient, the separated blood components may be subjected to an additional treatment to ensure the safety of the blood component intended for transfusion. Specifically, the collected blood components are treated to remove or otherwise inactivate virus and/or bacteria (“pathogens”) which may reside in the particular blood component. Many pathogen inactivation methods involve combining the blood component to be inactivated with a photosensitizer compound, which, when stimulated by light, acts on the pathogen, destroying its ability to replicate.

The steps of separating blood into components and pathogen reducing the components are not typically done together as part of one procedure. At some point in time after the blood is separated into components, the photosensitizer compound and blood component to be inactivated are typically added to a separate illumination bag to be exposed to light. Such procedure requires additional bags and additional sterile connections of bags, thus increasing the possibility for contamination during the multiple connections.

It would therefore be desirable to provide disposable sets for separating blood into components which includes a photosensitizer compound prepackaged within the sets to allow for easy preparation of such treatment-ready blood products. It is to the simplification of the blood separation and pathogen reduction process that the present invention is directed.

### SUMMARY OF THE INVENTION

This invention claims a pre-connected disposable set for blood separation and pathogen reduction comprising a separation vessel and at least one product bag pre-connected via a product collection conduit to the separation vessel. The at least one product bag may contain a photosensitizer, or there may be a second bag containing photosensitizer which is fluidly connected to the at least one product bag. There may also be an additional bag to collect whole blood fluidly connected to the separation vessel.

Also claimed is a method of using the above disposable set.

### BRIEF DESCRIPTION OF THE FIGURES

FIG. 1 is a schematic view of a set of separation and collection bags designed for cooperating with an automated apheresis apparatus.

FIG. 2 is a schematic view of another embodiment of the set of separation and collection bags shown in FIG. 1.

FIG. 3 is a schematic view of a set of separation and collection bags designed for cooperating with an automated whole blood separation apparatus.

FIG. 4 is a schematic view of another embodiment of the set of separation and collection bags shown in FIG. 3.

FIG. 5 is a schematic view of another set of separation and collection bags designed for cooperating with another automated whole blood separation apparatus.

FIG. 6 is a schematic view of another embodiment of the set of separation and collection bags shown in FIG. 5.

FIG. 7 shows an embodiment of this invention for treating the blood component to be inactivated with light.

### DETAILED DESCRIPTION OF THE INVENTION

A “photosensitizer” useful in this invention is defined as any compound which absorbs radiation at one or more defined wavelengths and subsequently utilizes the absorbed energy to carry out a chemical process.

Many photosensitizers may be used in this invention. Porphyrins, psoralens, quinacrines, and dyes such as methylene blue may all be used with this invention.

Endogenous photosensitizers may also be used. The term “endogenous” means naturally found in a human or mammalian body, either as a result of synthesis by the body or because of ingestion as an essential foodstuff (e.g. vitamins) or formation of metabolites and/or byproducts in vivo. When endogenous photosensitizers are used, particularly when such photosensitizers are not inherently toxic or do not yield toxic photoproducts after photoradiation, no removal or purification step is required after decontamination, and the decontaminated product can be directly returned to a patient's body or administered to a patient in need of its therapeutic effect.

Examples of such endogenous photosensitizers which may be used in this invention are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavin adenine dinucleotide [FAD]) and alloxazine mononucleotide (also known as flavin mononucleotide [FMN] and riboflavin-5-phosphate). The term “alloxazine” includes isoalloxazines.

Use of endogenous isoalloxazines as photosensitizers to inactivate blood and blood components are described in U.S. Pat. Nos. 6, 258,577 and 6,277,337 both issued to Goodrich et al., and herein incorporated by reference to the amount not inconsistent.

The amount of photosensitizer to be mixed with the blood and blood components to be inactivated will be an amount sufficient to adequately inactivate any pathogenic nucleic acids which may be present in the fluid, but less than a toxic (to the desired components) or insoluble amount. If riboflavin is used as the photosensitizer, it may be added to the fluid at a final concentration of between about 50-500 μM. Pathogenic nucleic acid includes any undesirable nucleic acid such as nucleic acid contained in white blood cells, bacteria or viruses. Nucleic acids include either deoxyribonucleic acid (DNA), ribonucleic acid (RNA) or both.

The blood or blood components to which the photosensitizer has been added is exposed to light of the appropriate wavelength to activate the photosensitizer and to cause substantially permanent damage to the pathogenic nucleic acids.

### Apheresis Disposable Set

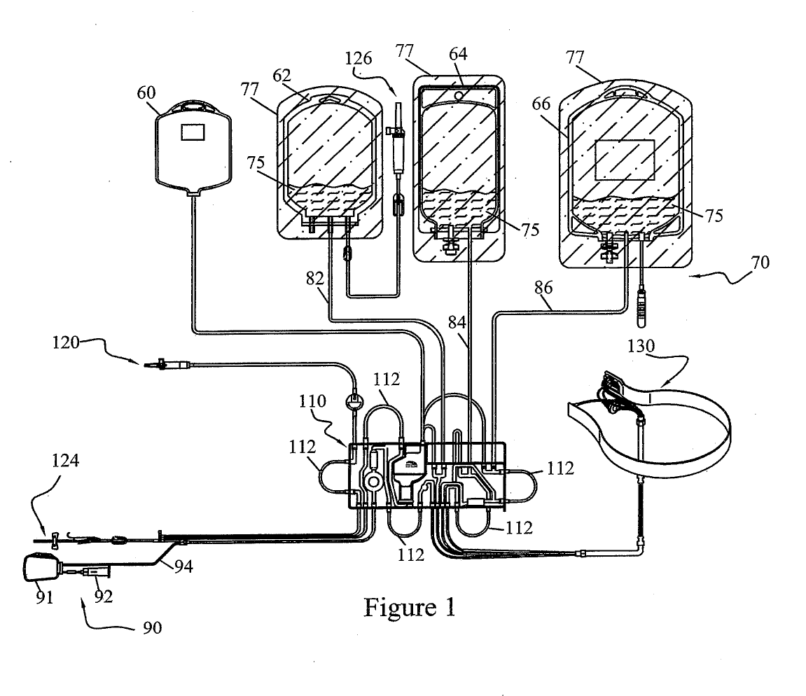

FIG. 1 shows a typical disposable set 70 for use with an apheresis machine. One such machine which may be used is the TRIMA® apheresis machine, available from Gambro BCT, Inc. (Lakewood, Colo., USA).

It should be noted that in the Figures, like elements are denoted using like numerals.

The disposable set 70 includes a separation vessel 130 for separating blood into various blood components. The set 70 also includes a fluid flow cassette 110 having peristaltic pump loops 112, a receive and return line 124 for receiving and returning blood and blood components to and from a donor, an anticoagulant line 120 for adding anticoagulant to the blood withdrawn from the donor, and a gas line 80 for receiving, into bag 60, gas purged from the system. The tubing set 70 and the separation vessel 130 are interconnected to create a closed disposable for a single use.

Various collection lines and bags for receiving separated blood components may also be part of the disposable set 70.

Red blood cell collection line or conduit 82 receives separated red blood cells from separation vessel 130 and leads to red blood cell product bag 62. A spike connection 126 is available to add storage solution (as but one example) to the red blood cells in product bag 62 if desired.

Plasma collection line or conduit 84 receives separated plasma from separation vessel 130 and leads to plasma product bag 64.

Platelet collection line 86 receives collected platelets from the separation vessel and leads to platelet product bag 66.

A sample set 90 may also be part of the disposable set 70. The sample set 90 includes line or conduit 94 through which a blood sample fluidly flows to sample bag 91. A blood sample may then be retrieved through sample device 92.

A solution containing the photosensitizer 75 could be placed in each of the product bags 62, 64 and 66 during manufacture and assembly of the bag set. The separated blood components would flow from the separation vessel 130 into the product bags which already contain the photosensitizer 75.

To prevent degradation of the photoactive material in the product bags before use, as well as to prevent evaporation of the photoactive solution during storage of the disposable set, the product bags could be contained within a removable opaque overwrap 77 which would prevent ambient light from activating the photoactive material before the blood product is added. The particular composition of the light filtering material will vary according to the light sensitivity spectrum of the photoactive material being used. If riboflavin is the photosensitizer being used, the overwrap 77 is made of foil.

By having the photosensitizer already in the product bags or in a second bag preconnected to the product bags, the steps of connecting an illumination bag to each product bag, connecting a bag containing the photosensitizer to the illumination bag, flowing the product to be inactivated from the product bag into the illumination bag, and flowing the photosensitizer solution into the product in the illumination bag would all be eliminated, reducing the likelihood of contamination during the multiple connections.

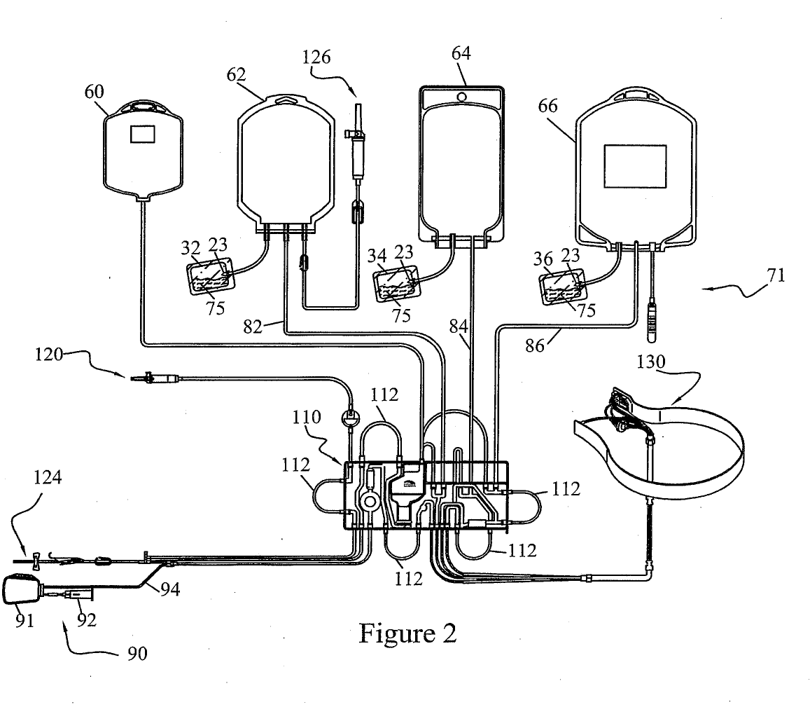

In another embodiment of a blood separation and pathogen reduction disposable set 71 shown in FIG. 2, bags 32, 34 and 36 containing riboflavin solution 75 could be fluidly connected to each product bag 62, 64 and 66 respectively by a conduit. The bags containing the riboflavin solution could be wrapped in a protective foil overwrap to prevent degradation of the photosensitizer by ambient light and prevent evaporation of the solution through the bag during storage of the disposable set.

After the product is collected in each bag, the frangible connectors 23 located in each riboflavin solution bag 32, 34 and 36 could be broken to allow the riboflavin solution to drain into the product bag. The product bag could then be illuminated.

In another embodiment, a bag containing the photoactive material could be attached to the disposable set 70 through spike port 120 (as but one example), flowed through the fluid flow cassette 110, the separation vessel 130, and into the product bag(s) 62, 64, 66 after the product(s) have been collected.

Although as shown in FIGS. 1 and 2 there are three product bags to collect platelets, plasma and red blood cells, it should be noted that only one product bag could be pre-connected to the separation vessel via a product conduit, to collect only one type of blood component. There could also be two product bags pre-connected to the separation vessel via product conduits to collect two types of blood components.

### Automatic Whole Blood Processing Disposable Set

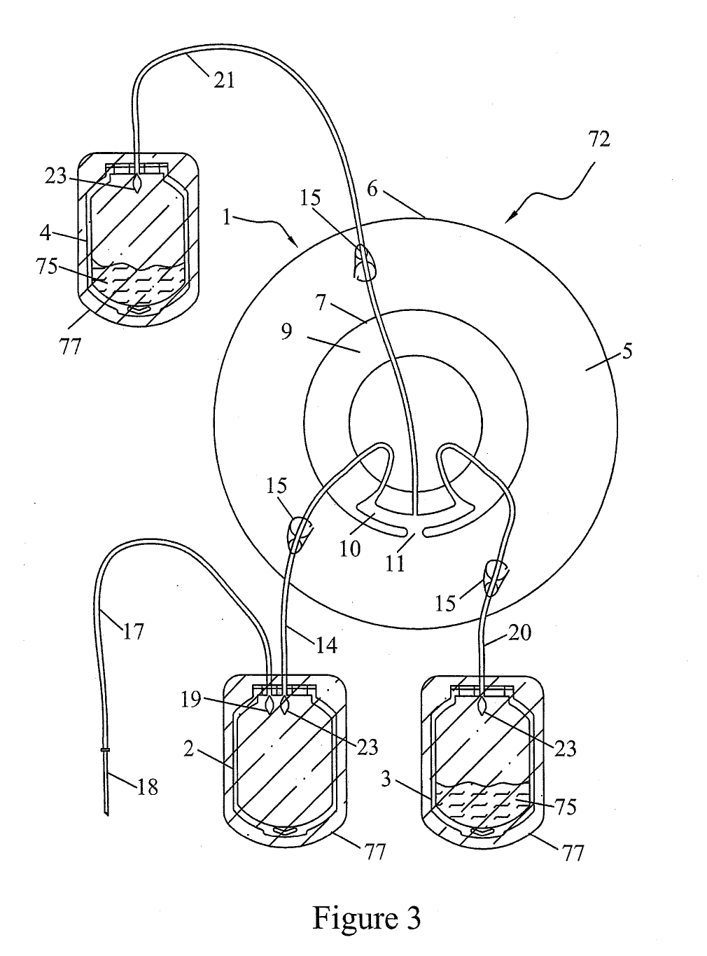

FIG. 3 shows a disposable bag set 72 for use in the separation and collection of previously collected whole blood into a plasma product, a platelet product and a red blood cell product using an automated whole blood separation device. The bag set can be used with an automated whole blood separation device, using a machine such as the Atreus® machine, manufactured by Gambro BCT, Inc. (Lakewood, Colo., USA.) and described in PCT/US2006/031492. This set 72 comprises a separation vessel which may be a bag 1 for separating whole blood into various blood components, and three product collection bags 2, 3 and 4.

The separation bag 1 comprises an annular separation chamber 5 having a substantially circular outer edge 6 and an inner circular edge 7. The separation bag 1 further comprises a semi-flexible disk-shaped connecting element 9 that is connected to the inner edge 7 of the annular chamber 5. The disk-shaped connecting element 9 comprises a distribution channel 10 embedded therein, which communicates through a passage 11 with the annular chamber 5. The distribution channel 10 substantially extends along an arc of the circle.

The first product bag 2 has two purposes and is successively used as both a whole blood collection bag and as a red blood cell component bag. The first product bag is intended for initially receiving a volume of whole blood from a donor (usually about 450 ml) before the separation process, and subsequently the red blood cell component during the separation process. The first product bag 2 is flat and substantially rectangular. It is connected to the separation bag 1 by a first transfer tube 14, fitted with a clamp 15. The first transfer tube 14 has a first end connected to the upper edge of the first product bag 2 and a second end connected to a first end of the distribution channel 10.

Anticoagulant is typically added to the first product bag 2 before whole blood is added. Typically about 63 ml of anticoagulant solution is added to a whole blood donation of about 450 ml. A frangible pin 23 removable from within the first product bag 2 prevents the anticoagulant from flowing out of the first product bag 2 into the separation bag 1 through the first transfer tube 14. The frangible pin 23 could also be located within transfer tube 14.

Whole blood collection tube 17 is connected at one end to the upper edge of the first product bag 2 and comprises, at the other end, a needle 18. A frangible pin 19 removable from within the first product bag 2 plugs the downstream end of the collection tube 17 and prevents the anticoagulant solution from flowing out of the first product bag 2 through the collection tube 17.

The second product bag 3 is intended for receiving a plasma component. It is flat and substantially rectangular. It is connected by a second transfer tube 20 to the separation bag 1. The second transfer tube 20, which is fitted with a clamp 15, has a first end connected to the upper edge of the second product bag 3 and a second end connected to a second end of the distribution channel 10.

The third product bag 4 is intended for receiving a platelet cell component. It is also flat and substantially rectangular. It is connected by a third transfer tube 21 to the separation bag 1. The third transfer tube 21 has a first end connected to the upper edge of the third product bag 4 and a second end that is connected to the distribution channel 10 so as to face the passage 11 between the distribution channel 10 and the separation chamber 5. The third transfer tube 21 is also fitted with a clamp 15.

If leukoreduction of the red blood cell product is desired, the first product bag 2 may be connected to a leukoreduction filter (not shown). The filter could be located in line with tubing 14.

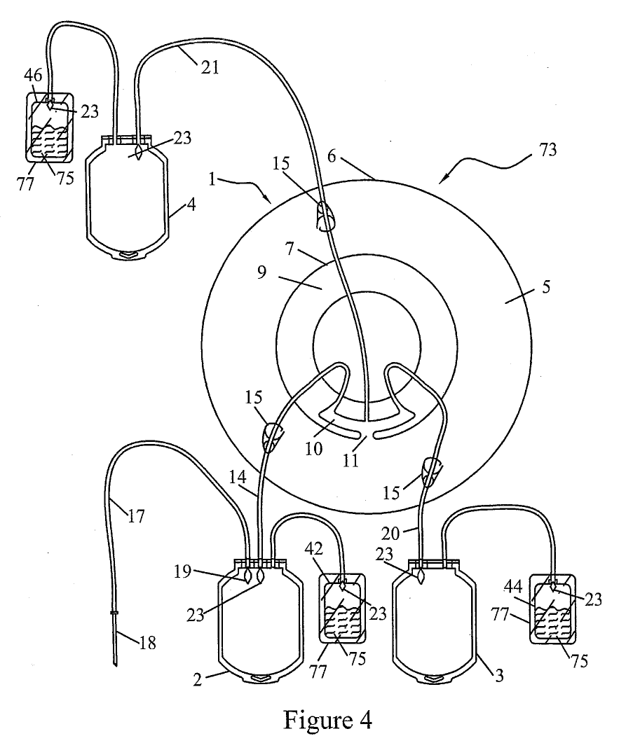

A solution containing the photosensitizer 75 could be placed in at least product bags 3 and 4 during manufacture and assembly of the bag set. The separated blood components would flow from the separation vessel 1 into the product bags 3, 4 which would already contain the photosensitizer 75. Although not shown in FIG. 3 (but see FIG. 4), a second bag containing photosensitizer solution could be fluidly connected by a conduit to bag 2. After the separated red blood cell component is returned to bag 2, the photosensitizer solution could be added. Frangible pins 23 would prevent the flow of photosensitizer 75 from the product bags 2, 3, 4 into the separation bag 1.

To prevent degradation of the photoactive material 75 in the product bags 2, 3 and 4 before use, the product bags could be contained within a removable opaque overwrap 77 which would prevent ambient light from activating the photoactive material as well as preventing evaporation of the photoactive solution during storage of the tubing set. The particular composition of the light filtering material will vary according to the light sensitivity spectrum of the photoactive material being used. If riboflavin is the photosensitizer being used, the overwrap 77 is made of foil.

By having the photosensitizer either in the product bags or in a second bag preconnected to the product bags, the steps of connecting an illumination bag to each product bag, connecting a bag containing the photochemical to the illumination bag, flowing the product into the illumination bag, and flowing the photochemical solution into the product in the illumination bag would all be eliminated, reducing the likelihood of contamination during the multiple connections.

In another embodiment of a blood separation and pathogen reduction set 73 shown in FIG. 4, bags 42, 44 and 46 containing riboflavin solution 75 could be fluidly connected to each product bag 2, 3 and 4 respectively. The bags containing the riboflavin solution 75 could be wrapped in a protective foil overwrap 77 to prevent degradation of the photosensitizer by ambient light and prevent evaporation of the solution through the bag.

After the product is collected in each bag, the frangible connectors 23 located in each riboflavin solution bag 42, 44 and 46 could be broken to allow the riboflavin solution to drain into the product bag/s. The product bag/s could then be illuminated.

In another embodiment, an additional bag (not shown) containing the photosensitizer may also be attached to the separation bag 1. The photosensitizer may be metered from the additional bag through the separation bag into the product bags after the separated blood components have been collected in the product bags. Alternatively, the photosensitizer could be metered into the separation bag before the separation process. The separated blood products would already contain photosensitizer when flowed into the product bags.

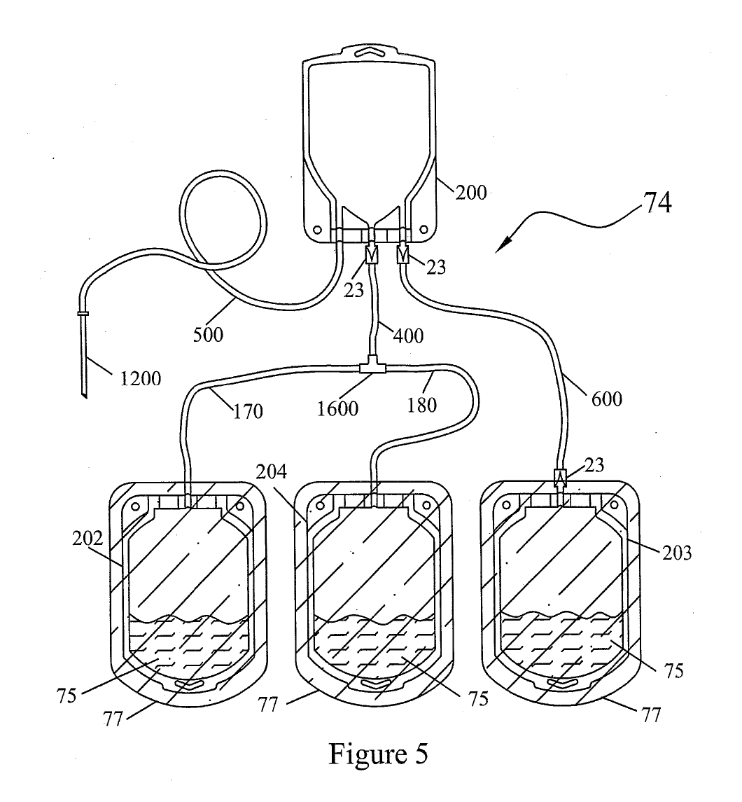

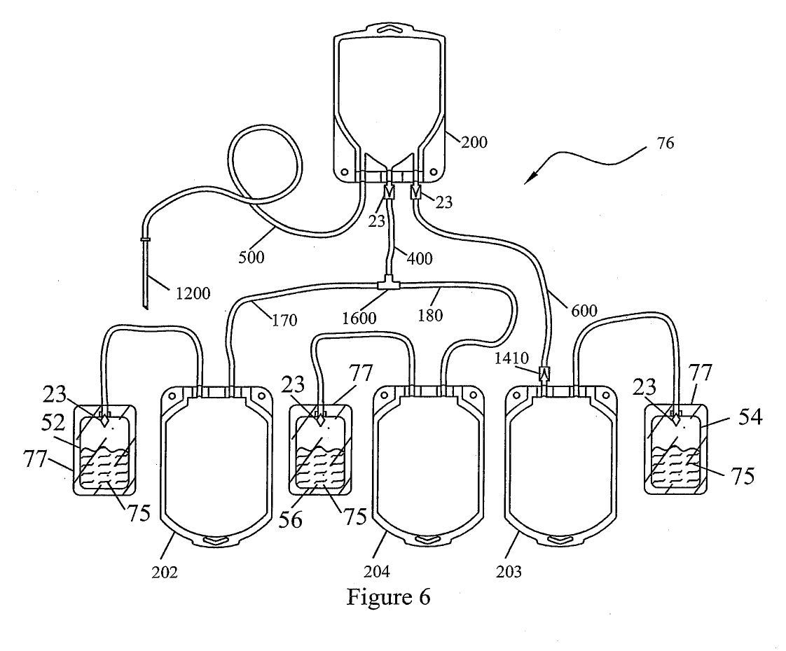

Although as shown in FIGS. 5 and 6 there are three product bags to collect platelets, plasma and red blood cells, it should be noted that only one product bag could be pre-connected to the separation vessel via a product conduit, to collect only one type of blood component. There could also be two product bags pre-connected to the separation vessel via product conduits to collect two types of blood components.

### Self-Balancing Bag Set

Another disposable bag set 74, which may be used with another automated whole blood separation device which is self balancing, is shown in FIG. 5. This disposable set may be used with another automated whole blood separation machine which is self balancing, and described in PCT/US2006/021674. This bag set comprises a flexible separation bag 200 and three flexible blood product bags 202, 203, 204 connected thereto.

The separation bag 200 has at least two purposes, and is successively used as both a whole blood collection bag and as a separation bag. It is intended for initially receiving a discrete volume of whole blood from a donor (usually about 450 ml) and to be used later as a separation chamber in a separation apparatus. The separation bag 200 is flat and generally rectangular. It is made of two rectangular sheets of plastic material that are welded together so as to define therebetween an interior space having a main rectangular portion connected to a triangular top downstream portion. A first tube 400 is connected to the tip of the triangular portion, and second and third tubes 500, 600 are connected to both lateral edges of the triangular portion, respectively. The proximal ends of the three tubes 400, 500, 600 are embedded between the two sheets of plastic material so as to be parallel.

The separation bag 200 may initially contain a volume of anti-coagulant solution, and the first and third tubes 400, 600 are fitted at their proximal ends with frangible connectors 23 respectively, blocking the flow of fluid therethrough.

The second tube 500 is a collection tube having a needle 1200 connected to its distal end. At the beginning of a blood donation, the needle 1200 is inserted in the vein of a donor and blood flows into the collection (separation) bag 200. After a desired volume of blood has been collected in the collection (separation) bag 200, the collection tube 500 is sealed and cut.

The first product bag 202 is intended for receiving a separated plasma component. It is flat and substantially rectangular. It is connected to the distal end of the first tube 400.

The second product bag 203 is intended for receiving a separated red blood cell component. It is flat and substantially rectangular. It is connected to the distal end of the third tube 600. The red blood cell collection bag 203 may contain a volume of storage solution for red blood cells, and the third tube 600 is fitted at its distal end with a frangible connector 23 blocking a liquid flow therethrough. An in-line leukoreduction filter (not shown) may also be attached in tubing line 600 if leukoreduction is desired.

The third product bag 204 is intended to receive a platelet component, and a T-shaped three-way connector 1600 having its leg connected by the first tube 400 to the separation bag 200, a first arm connected by a fourth tube 170 to the collected plasma bag 202, and a second arm connected by a fifth tube 180 to the collected platelet bag 204. Like the plasma and red blood cell bags 202, 203, the platelet bag 204 is flat and substantially rectangular.

The photosensitizer 75 could be placed in each of the product bags 202, 203 and 204 during manufacture and assembly of the bag set. The separated blood components could flow from the separation vessel 200 into the storage bags which would already contain the photochemical.

In another embodiment of an automated whole blood separation device 76 shown in FIG. 6, bags 52, 54, 56 containing riboflavin solution 75 could be fluidly connected to each product bag 202, 203 and 204 respectively. The bags containing the riboflavin solution 75 could be wrapped in a protective foil overwrap 77 to prevent degradation of the photosensitizer by ambient light and prevent evaporation of the solution through the bag.

After the product is collected in each bag, the frangible connectors 23 located in each riboflavin solution bag 52, 54, 56 could be broken to allow the riboflavin solution to drain into the product bag/s. The product bag/s could then be illuminated.

By having the photosensitizer already in the product bags or in a second bag preconnected to the product bags, the steps of connecting an illumination bag to each product bag, connecting a bag containing the photochemical to the illumination bag, flowing the product into the illumination bag, and flowing the photochemical solution into the product in the illumination bag would all be eliminated, reducing the likelihood of contamination during the multiple connections.

An additional bag (not shown) containing the photosensitizer may also be attached to the separation bag 200. The photosensitizer may be metered from the additional bag through the separation bag 200 into the product bags after the separated blood components have been collected in the product bags. Alternatively, the photosensitizer could be metered into the separation bag before the separation process. The separated blood products would already contain the photosensitizer when flowed into the product bags.

In a method of using the disposable separation and pathogen inactivation sets of the present invention, once the separated blood products have been directed from the separation vessel into their respective product bags containing photosensitizer, the bag(s) may be sterilely removed and sealed off from the rest of the bag set using any known methods.

If an opaque overwrap is part of the tubing set, once the separated blood components have been collected in the product bags, the removable opaque overwrap is removed from the product bags immediately before the blood component is illuminated.

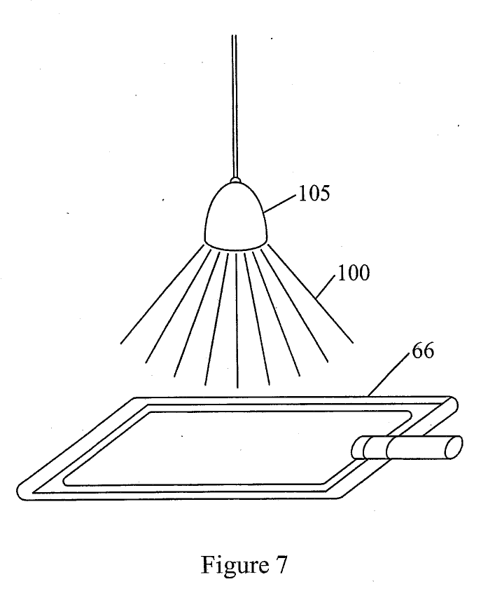

As shown in FIG. 7, after the bag (designated as element 66 for example purposes only) containing the platelet component to be inactivated is removed from the rest of the disposable bag set 70 (not shown and given as but one example), the opaque overwrap 77 is removed, and the bag is immediately exposed to radiation 100 from a light source 105 for a time sufficient to inactivate any pathogens which may be present in the blood product, but less than an amount which would cause damage to the blood product.

If the photosensitizer is added to the product bag from the bag containing photosensitizer, there would be no opaque overwrap to be removed from the product bag.

The pathogen inactivated blood product may then be stored for a period of time before being transfused into a recipient, or may be transfused into a recipient immediately after the pathogen inactivation procedure.

It is understood for the purposes of this disclosure that various changes and modifications may be made to the invention that are well within the scope of the invention. Numerous other changes may be made which will readily suggest themselves to those skilled in the art and which are encompassed in the spirit of the methods disclosed herein.

1. A method of pathogen inactivating blood components which may contain pathogens comprising:
separating whole blood in a separation vessel into at least one component;
flowing the at least one separated blood component from the separation vessel into a product bag containing a photosensitizer pre-connected to the separation vessel
wherein the product bag is covered by a removable opaque overwrap;
removing the removable opaque overwrap; and
illuminating the at least one separated blood component and photosensitizer for a time sufficient to inactivate any pathogens which may be present in the blood component but less than a time which would cause damage to the blood component.

2. The method of claim 1 further comprising the step of removing the product bag from the separation vessel before the illuminating step.

3. A method of pathogen inactivating blood components which may contain pathogens comprising:
separating whole blood in a separation vessel into at least one blood component;
flowing the at least one separated blood component from the separation vessel into a product bag pre-connected to the separation vessel;
flowing a photosensitizer from a bag pre-connected to the product bag into the product bag; and
illuminating the product bag for a time sufficient to inactivate any pathogens which may be present in the blood component but less than a time which would cause damage to the blood component.

4. The method of claim 3 further comprising the step of removing the product bag from the separation vessel before the illuminating step.

5. The method of claim 3 further comprising the step of removing the bag which had contained the photo sensitizer from the product bag before the illuminating step.
